(19) United States
(12) Patent Application Publication
(10) Pub. No.: US 2007/0274909 A1
(43) Pub. Date: Nov. 29, 2007

(54) Radiation Therapy and Medical Imaging Using Uv Emitting Nanoparticles

(75) Inventors: Thomas Justel, Witten (DE); Claus Feldmann, Ettlingen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONIC, N.V., EINDHOVEN (NL)

(21) Appl. No.: 10/596,440

(22) PCT Filed: Dec. 9, 2004
(86) PCT No.: PCT/IB04/52725

§ 371 Date: Feb. 20, 2007

(30) Foreign Application Priority Data
Dec. 17, 2003 (EP) 03104756.6

(51) Int. Cl. A61K 41/00; A61K 49/00; A61K 49/04; A61P 35/00

(52) U.S. Cl. 424/1.53; 424/1.49; 977/915; 977/928

(57) ABSTRACT

The invention relates to UV emitting nanoparticles for radiation therapy purposes. If the nanoparticles are brought indirectly or directly to the diseased tissue, excitation with high energy radiation leads to VUV or UV-C emission. This UV radiation is absorbed by the surrounding organic matrix, resulting in decomposition of the material. The nanoparticles can also be modified by attaching antibodies to the particles by chemical linking or coating. Preferably these antibodies bind specifically to the cell membrane of cancer cells leading to a localised destruction of diseased tissue with a high efficacy and a lower level of destruction of surrounding healthy tissue. Endoscopic detection of the UV emission can be used as a medical imaging technique to locate and study diseased tissue.

The present invention relates to materials and methods used in radiation therapy or medical imaging. More specifically, the invention is related to nanoparticles used in treatment of diseased tissue or for imaging tissue.

Imaging techniques such as X-ray computer tomography (CT), positron emission tomography (PET), single photon emission tomography (SPECT), nuclear spin magnetic resonance tomography (MRI), ultra sound techniques, are widely used in medical diagnostics. Nevertheless, most of these tomographic methods require a large financial investment both when the system is purchased and for paying an expert to perform the measurements and interpret the results. Optical techniques have the advantage that they are often cheaper and that they furthermore allow easier interpretation of the results.

Diseased tissue or cancerous tumours are often treated by using ionising radiation, a process that is known as radiation therapy. Radiation therapy for cancer, which typically uses electromagnetic radiation with energies of a few keV to a few MeV, typically works by attacking rapidly growing cells with highly penetrating ionising radiation. The use of x-rays is attractive due to its ability to penetrate deeply into tissue, especially if the diseased tissue is bone or other dense or opaque structures or if the diseased tissue is located within bone or other dense or opaque structures. Unfortunately, using rapid growth as the sole targeting criterion does not limit the effects of such treatment solely to cancer cells. Consequently, also healthy tissue will be damaged.

As a result, many methods have been developed for delivery of the ionising radiation to the site of the cancerous tumour so as to limit the effects of such radiation to the general area of the cancerous tissue. However, since healthy tissue and cancerous tissue typically have a similar biological response to radiation, a need exists to improve the potency of, or biological response to, the delivered radiation within and in the vicinity of the tumour, while not affecting the surrounding healthy tissue. A known method which allows to reduce the X-ray dose is to further sensitise tumours to radiation by reducing the amount of competing metabolites and thus favouring specific metabolites which are more sensitive to the radiation.

An alternative approach to radiation therapy is the application of radionuclides, which is in particular useful for the treatment of diseased tissue or tumours located deep in the patient's body or located within bone or other opaque structures. If e.g. ²¹²Bi³⁺ is used, the bismuth particle decays into a thallium particle, thereby emitting an alpha-particle
²¹²Bi→α+²⁰⁸Tl

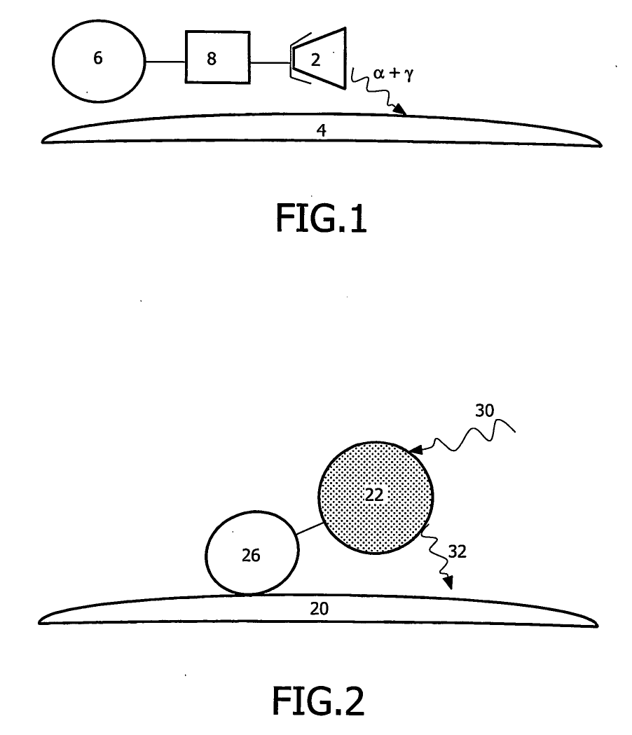

To achieve high specificity to cancer cells, the radionuclide cations are chelated, i.e. tightly bound, by an organic moiety, e.g. Ethylene Diamine Tetra acetic Acid (EDTA), which is conjugated to an antibody with a high specificity to cancer cells. FIG. 1 shows a schematic mechanism of a therapy approach for the treatment of cancer by using radioactive nuclides. A radioactive nuclide 2, e.g. ²¹²Bi³⁺, decays in the surrounding of the cancer cell membrane 4. Thereto, the radioactive nuclide 2 is bound to an antibody 6, which has high specificity for these cancer cells, by an organic moiety 8, e.g. methylene leucine Leu-CH₂ or Leucine. However, the problems of this approach are the toxicity of the agents to be injected into the patient and the short half-life of useful radionuclides, e.g. 1 hour for ²¹²Bi, 13.3 hours for ¹²³I and 7 hours for ²¹²At.

As an alternative to the use of ionising radiation, photodynamic therapy (PDT) has been developed. In PDT, a photosensitive agent is combined with a radiation source, emitting non-ionising, optical radiation, to produce a therapeutic response in diseased tissue. In PDT, a distinct concentration of a photosensitive agent is to be located in the diseased tissue and not in the healthy surrounding tissue. This is performed either through natural processes or via localised application by injection. To enhance the specificity of the photosensitive agent to diseased tissue it is commonly conjugated to a targeting moiety, which can be an antibody or an organic functional group showing higher binding constants to cancer cells/tissue than to healthy cells/tissue. This provides an additional level of specificity relative to that achievable through standard radiation therapy since PDT is effective only where the sensitiser is present in tissue. As a result, damage to surrounding and healthy tissue can be avoided by controlling the distribution of the agent. Unfortunately, when using conventional methods for the illumination step in PDT, the light required for such treatment is unable to penetrate deeply into tissue. In addition, the physician has only restricted spatial control of the treatment site which is troublesome if the diseased tumour is located deeply in the body.

U.S. Pat. No. 6,530,944 by West et al. relates to medical imaging and localised treatment of cancer using heat. Cells are killed by the induction of heat generated from nanoparticles after irradiation with infrared light. These nanoparticles can be e.g. silica doped with rare earth emitters. The therapeutic method presented comprises the delivery of these infrared emitting nanoparticles to the diseased tissue. This can e.g. be done by binding the nanoparticle to an antibody, which has high specificity for the diseased tissue. The nanoparticle is then excited preferably using infrared radiation with a wavelength from 580 nm up to 1400 nm, upon which it emits heat. The cells in the surrounding of the nanoparticle are killed due to denaturation of cellular proteins by the generated heat. This technique thus comprises the use of certain compounds to convert infrared radiation into another energy with the purpose to damage living cells. Furthermore, visible and near-infrared emitting nanoparticles are used in spin-coating and photolithography applications. In that case, the particles are made of LaF₃ and LaPO₄ doped with the luminescent trivalent lanthanide ions Eu³⁺, Nd³⁺, Er³⁺, Pr³⁺, Ho³⁺ or Yb³⁺ as this allows dispersability in organic solvents.

Nevertheless, U.S. Pat. No. 6,530,944 has some disadvantages. The penetration depth of radiation into organic matter increases with decreasing energy from the visible to the IR, deep red and near IR is hardly absorbed. Thus, the generated IR radiation has a high penetration depth. Therefore, it is difficult to limit the generated IR radiation to the location of the diseased tissue and hence, there is a possibility that the radiation also reaches the healthy tissue.

It is an object of the present invention to provide means and methods for localtherapy, possibly located deep in the human body, while preferably limiting the amount of damage to healthy tissue.

It is another object of the present invention to provide means and methods for medical imaging, possibly located deep in the human body, while limiting the amount of damage to healthy tissue.

The above objective is accomplished by materials, methods and means for therapeutic treatment and medical imaging according to the present invention.

The present invention provides nanoparticles for use in imaging or in a radiation treatment of bilogical material such as in radiation therapy, e.g. of diseased tissue. The nanoparticles comprises a VUV or UV-C emitting material which absorbs high energy radiation and emits VUV or UV-C radiation and are conjugated to a bio-target specific agent such as a microorganism, e.g. parasite, biomolecule, e.g. protein, DNA, RNA, cell, cell organelle or tissue target agent. Preferably the bio-target is a therapeutically relevant target. The high energy radiation may be X-rays. The bio-target specific agents may for example be antibodies or antibody fragments, which may have a specificity for the relevant bio-target, e.g. a diseased tissue.

Furthermore, the UV emitting material of the nanoparticles may be provided with a covering layer. The covering layer may prevent hydrolysis of the UV emitting material or enhance entry through cell membranes, etc.

The VUV or UV-C emitting material may be one or more substances selected from the group M₂SiO₅:X, MAlO₃:X, M₃Al₅O₁₂:X, MPO₄:X, MBO₃:X, MB₃O₆:X with M=Y, La, Gd, Lu, and X=Pr, Ce, Bi, Nd or any of MM′O₃:X with M=Y, La, Gd, Lu, M′=Y, La, Gd, Lu, Bi and X=Pr, Ce, Bi or any of MSO₄:Z with M=Sr, Ca and Z=Nd, Pr, Ce, Pb or any of LuPO₄:Nd, YPO₄:Nd, LaPO₄:Nd, LaPO₄:Pr, LuPO₄:Pr, YPO₄:Pr, YPO₄:Bi.

In a specific embodiment, the VUV or UV-C emitting material may be a trivalent phosphate.

In another embodiment, the nanoparticles may be doped with an activator. The activator may have a decay time shorter than 100 ns. In a specific embodiment, the activator may be Pr³⁺ or Nd³⁺.

The present invention furthermore provides the use of nanoparticles as an imaging agent or as a radiation treatment agent of biological material, e.g. as a radiation therapy agent for diseased tissue, the nanoparticles comprising a VUV or UV-C emitting material which absorbs high energy radiation and emits VUV or UV-C radiation. The use includes the manufacture of the agents. The high energy radiation may be X-rays. The nanoparticles may be conjugated to a bio-target specific agent such as a microorganism, e.g. parasite, biomolecule, e.g. protein, DNA, RNA, cell, cell organelle or tissue target agents. In one embodiment, the bio-target specific agents may be antibodies or antibody fragments and may have a specificity for the relevant bio-target, e.g. a diseased tissue.

In another embodiment, the UV emitting material of the nanoparticles may be provided with a covering layer. The covering layer may prevent hydrolysis of the UV emitting material.

The VUV or UV-C emitting material may be one or more substances selected from the group M₂SiO₅:X, MAlO₃:X, M₃Al₅O₁₂:X, MPO₄:X, MBO₃:X, MB₃O₆:X with M=Y, La, Gd, Lu, and X=Pr, Ce, Bi, Nd or any of MM′O₃:X with M=Y, La, Gd, Lu, M′=Y, La, Gd, Lu, Bi and X=Pr, Ce, Bi or any of MSO₄:Z with M=Sr, Ca and Z=Nd, Pr, Ce, Pb or any of LuPO₄:Nd, YPO₄:Nd, LaPO₄:Nd, LaPO₄:Pr, LuPO₄:Pr, YPO₄:Pr, YPO₄:Bi.

In a specific embodiment, the VUV or UV-C emitting material may be a trivalent phosphate.

In another embodiment, the nanoparticles may be doped with an acitvator. The activator may have a decay time shorter than 100 ns. In a specific embodiment, the activator may be Pr³⁺ or Nd³⁺.

The present invention also provides a method of treatment of a human or an animal patient by—providing nanoparticles according to the present invention,—administering the nanoparticles to the patient and—irradiating the patient with high energy radiation. Preferably, the radiation is localised to a specific part of the body.

It is an advantage of the present invention that the means and method may also be used for optical imaging by endoscopically detecting the emission of the nanoparticles. Furthermore, the present invention has an advantage in that it combines both medical imaging and therapeutic treatment in one technique.

It is furthermore an advantage of the present invention that the means for local treatment of microorganisms or cells, e.g. diseased tissue, has a high efficacy for destroying such microorganisms, cells or diseased tissue and a low toxicity. Furthermore, the means for local treatment of diseased tissue consist of cheap basic materials.

Although there has been constant improvement, change and evolution of therapeutic methods in this field, the present concepts are believed to represent substantial new and novel improvements, including departures from prior practices, resulting in the provision of more efficient, stable and reliable devices of this nature.

The teachings of the present invention permit the design of improved therapeutic methods and imaging methods for treatment of diseased tissue or cancerous tumours.

These and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

FIG. 1 is a schematic representation of a conventional method of treatment of cancer by using radioactive nuclides.

FIG. 2 shows a UV emitting nanoparticle conjugated to an antibody according to an embodiment of the present invention.

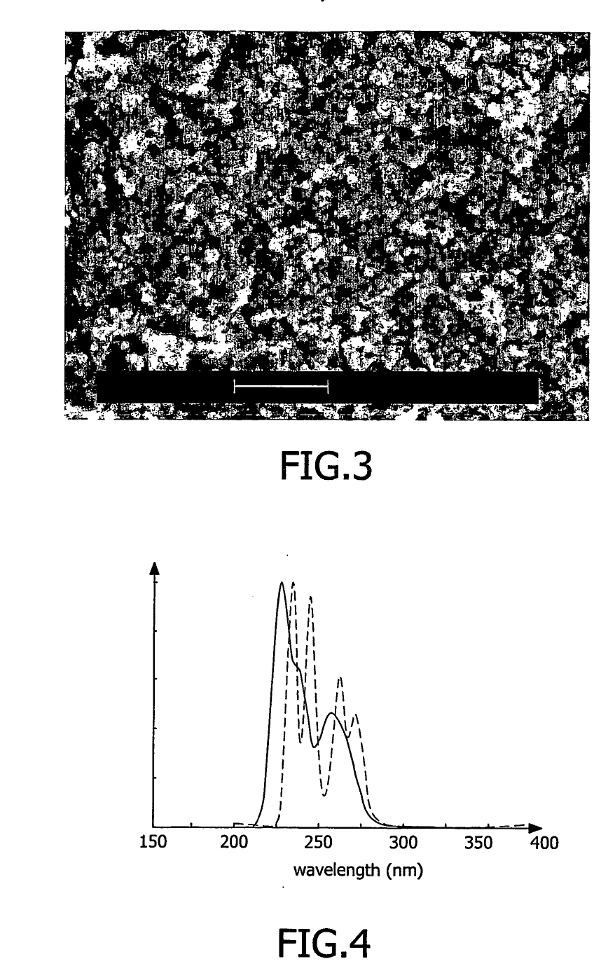

FIG. 3 shows a scanning electron microscopy picture of LaPO₄:Pr nanoparticles having a particle size of about 100 nm according to an embodiment of the present invention.

FIG. 4 is a graph of the emission intensity as a function of the wavelength for high energy excitation of LaPO₄:Pr (solid line) and YPO₄:Pr (dashed line) nanoparticles according to embodiments of the present invention.

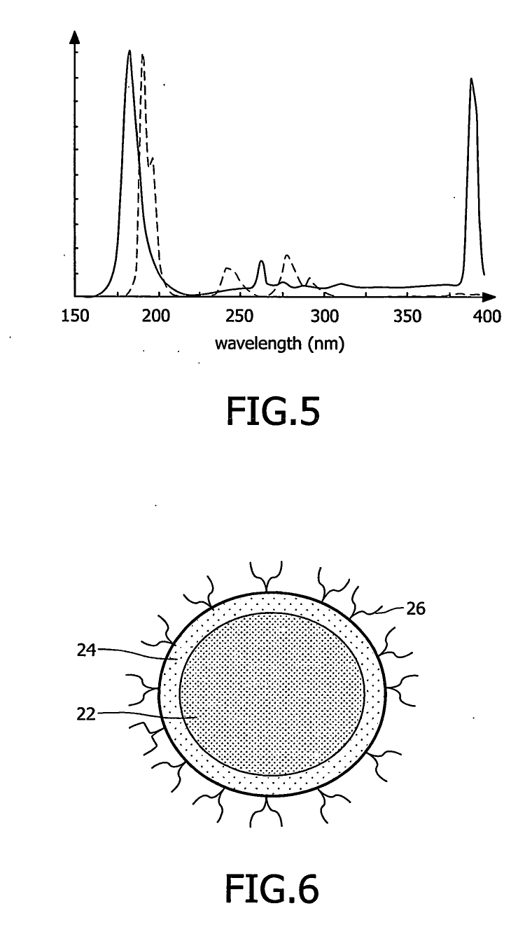

FIG. 5 is a graph of the emission intensity as a function of the wavelength for high energy excitation of LaPO₄:Nd (solid line) and YPO₄:Nd (dashed line) nanoparticles according to embodiments of the present invention.

FIG. 6 is a schematic representation of a method of treatment of cancer employing VUV emission under x-ray excitation of phosphate nanoparticles according to an embodiment of the present invention.

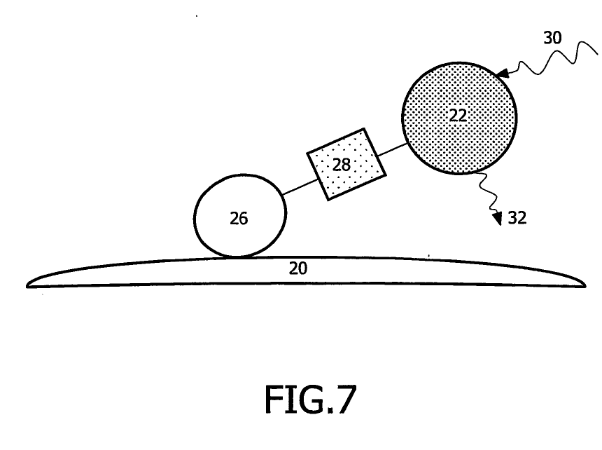

FIG. 7 shows a specific embodiment of a UV-emitting nanoparticle conjugated to an antibody according to an embodiment of the present invention.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In the following reference will be made to the treatment of a cell or tissue type, e.g. in cancer treatment. However, the present invention is not limited to this type of cell nor to this type of treatment but may have wide application in radiation treatment of any biological material and in radiation therapy and diagnosis and imaging, especially medical imaging.

Generally, there is a need to incapacitate or destroy certain bio-targets, e.g. in biological material such as food products, or in human or animal therapy. These bio-targets could be, for example, a diseased cell, e.g. a cancer cell, a microorganism, e.g. a parasite such as a nematode, a bacterium, a virus. For each of these bio-targets a agent can be provided which binds or associates itself with some specificity to that target. The specificity may be relative, i.e. relative to local biological material or tissue which does not belong to the biotarget. An example, is healthy tissue in the neighbourhood of diseased tissue. The biotarget agent should have specificity with respect to the biotarget, e.g. diseased cells while having a reduced or essentially no specificity to the healthy tissue. One good example of such a binding agent is a polyclonal or monoclonal antibody or fragment(s) thereof. Another suitable targeting agent could be a substance specifically ingested by a parasite. In accordance with one aspect of the present invention the bio-targeting agent is associated with, or bound to a material which emits radiation of a certain wavelength in the UV spectrum when irradiated with another type of radiation such as X-rays. The emitted UV radiation provides a local therapeutic effect, e.g. destroying a parasite or a diseased cell. The present invention does not exclude that healthy cells or tissue may be damaged in this process but the low penetration depth of the UV radiation reduces this damage to a minimum.

A therapeutic treatment in accordance with the present invention can be used for treatment of cancer, non-malignant tumours, auto-immune diseases, etc. as indicated above. An improved cancer therapy approach is preferably based on sensitising agents with a low toxicity to obtain an improved light-to-dark cytotoxicity ratio and the corresponding excitation source should have a sufficiently large penetration depth to achieve therapeutic effect for diseased tissue that is located within bone or deeply in the human body. Furthermore, the type of excitation source or the amount of energy should be such that damaging by the excitation source is limited. Achieving these conflicting requirements has proved elusive.

With cancer, the most general medical definition of cancer is referred to wherein the disease is characterised by uncontrolled growth and spread of abnormal cells. Non-malignant tumours refer to benign tumours which remain in that part of the body in which they start growing, but which may exert pressure on other body parts. Auto-immune diseases are diseases wherein the immune system, which is a complicated network of cells and cell components, mistakenly attacks cells, tissues and/or organs of a person's own body. An example of such a disease is multiple sclerosis. Cancerous tumours as well as benign tumours and cells affected by auto-immune diseases will be referred to as diseased tissue.

The therapeutic method of this invention may be used either in vitro or in vivo. The methods may be applied both to the human body and to animals and also to tissue or organs removed from such animals, e.g. an organ such as a kidney or liver which is to be transplanted.

In a first embodiment according to the present invention, a UV-emitting material is used for radiation therapy of diseased tissue 20. In this embodiment, the material comprises nanoparticles 22 which typically have one dimension such as a diameter in the range from 1 nm to 100 nm. Although the nanoparticles 22 are represented in the drawings by spheres, the nanoparticles 22 may have any suitable shape including quadrilateral, cylindrical, rod-like, or oval or a more irregular shape and morphology. The nanoparticles 22 typically comprise a host matrix which is intentionally doped. The energy levels of the dopant atoms or the clusters of dopant atoms can be strongly influenced by the surrounding host material. In accordance with an aspect of the present invention, host materials and dopants are selected such that the doped host matrix emits light in the UV region. In principle, the particles 22 can also comprise non-intentionally doped host materials as long as efficient emission in the UV or VUV region is achieved upon excitation. The latter could be e.g. obtained by recombination emission. The UV-C region is defined as the wavelength region 280 nm-100 nm whereas the VUV region (Vacuum Ultra Violet) is defined as the wavelength region 200 nm-10 nm.

The nanoparticles 22 are conjugated to target agents 26 such as antibodies, antibody fragments (FAB fragments) or an organic functional group showing higher binding constants to the target microorganism/cells/tissue etc. than to healthy cells/tissue. The antibodies or antibody fragments are preferably specific for the bio-target, e.g. diseased tissue 20 like for example cancer cells (FIG. 2). It is not necessary that the target agents are strongly specific to the diseased cells provided they bind to the diseased more preferably than healthy cells in the same region of the body or organ. The nanoparticles 22 can then be provided to the patient e.g. by injection into the blood, administration to the digestive system. When the nanoparticles 22 conjugated to the target agents, e.g. antibodies 26, they are spread through the human body, and the target agents, e.g. the antibodies 26 bind to the diseased tissue 20, e.g. by specific antibody-antigen reactions, leading to an increased nanoparticle 22 content and density in the region of the diseased tissue or tumour 20. This binding can occur either on the surface of the cells and/or tissue 20, e.g. on cell membranes, or to cell interior sites. The target agents such as the antibodies 26 can be either chemically bound to the nanoparticle 22 or a layer of target agents, e.g. antibodies 26 can be coated on the surface of the nanoparticle 22. A non-limiting list of examples of antibodies 26 and the corresponding specific diseases they are used for are given in table 1.

TABLE 1

| Antibody | Disease |
|---|---|
| Trastuzumab | Breast cancer |
| Rituximab | Non-Hodgkin Lymphoma |
| Alemtuzumab | Chronical lymphocytic |
| Gemtuzumab | Acute myelogenous |
| Edrecolomab | Intestinal cancer |
| Ibritumomab | Non-Hodgkin Lymphoma |
| Cetuximab | Intestinal cancer |
| Tositumomab | Non-Hodgkin Lymphoma |
| Epratulumab | Non-Hodgkin Lymphoma |
| Bevacizumab | Bronchopulmonary cancer |
| Anti-DC33 | Acute myelogenous |
| Pemtumomab | Overy cancer and Gastric |
| Mittumomab | Bronchopulmonary cancer |
| Anti-MUC 1 | Adenocarcinoma |
| Anti-CEA | Adenocarcinoma |

Besides antibodies 26, the nanoparticle 22 can also be conjugated to proteins that can enter through the cell membrane. Alternatively, antisense DNA may be used to target specific DNA or RNA sequences known to be present in diseased cells.

By absorption of energy from an internal or an external source, the nanoparticles 22 used according to the present invention emit VUV or UV-C radiation. As an internal source a nanoparticle material that comprises radioactive elements, as for example YPO₄:Pr, whereby Y, P or Pr is partly replaced by a radioactive isotope such as ³²P, ⁹⁰Y, ⁸⁸Y or ¹⁴³Pr, may be used. This yields self activation of the UV-C luminescence. A suitable external source is an X-ray source which has the required penetration depth for the location of the diseased cells in the body, e.g. with an energy higher than 7 keV. The X-rays are absorbed by the nanoparticles and the energy is re-emitted as UV light. Devices that may be used are for example X-ray tubes (Bremsstrahlung+Cu or Mo K, L-lines), ⁶⁰Co sources (2.82 MeV) or synchrotrons providing monochromatic and tunable X- to γ-rays. The emitted radiation from the nanoparticles is absorbed by the organic matrix of the surrounding diseased cells 20, resulting in the decomposition of this organic matter, finally yielding cell death. As discussed above, the wavelength region of the emission, according to this invention, typically has an upper limit of 280 nm. This leads to a limited penetration depth into the surrounding tissue, which is favourable as healthy tissue adjacent to the diseased tissue 20 suffers less damage. Moreover, the corresponding energy for photons with a wavelength smaller than 280 nm is necessary to obtain an effective therapeutic result. Photons with a wavelength below 280 nm are efficiently absorbed by RNA and DNA, while photons with a wavelength below 190 nm are absorbed by water molecules. The typical penetration depth of 190 nm photons in water is about 1 cm. Radiation between 190 nm and 280 nm is, at least partly, absorbed by amino acids. The absorption of photons due to DNA or RNA results in their cleavage, which disturbs the transcription and translation process in the cell. Absorption of photons by water yields OH- and H-radicals, H₂O→OH*+H*

which leads e.g. to the oxidative decomposition of proteins in the cytoplasm. Both processes inhibit cell growth or even kill exposed cells. The VUV/UV-C radiation thus is harmful and has a high photochemical efficiency. The effect is limited to those cells, which are adjacent the nanoparticles 22. The high efficacy of UV-C and VUV radiation to harm organic matter is an advantage compared to e.g. standard radiation therapy.

A non-limiting list of nanoparticle 22 materials emitting in the wavelength region useful in the method of the present invention is given in table 2. For some specific examples, the wavelength of the highest emission peak in the useful UV region is given in column 3.

TABLE 2

| Host material | Dopant | Emission |
|---|---|---|
| M₂SiO₅ | Pr, Ce, Bi | UV |
| (M = Y, La, Gd, Lu) | | |
| MAlO₃ | Pr, Ce, Bi | UV |
| (M = Y, La, Gd, Lu) | | |
| MM′O₃ | Pr, Ce | UV |
| (M/M′ = Y, La, Gd, Lu) | | |
| M₃Al₅O₁₂ | Bi, Pr, | UV |
| (M = Y, La, Gd, Lu,) | | |
| MPO₄ | Pr, Ce, Bi, Nd | UV |
| (M = Y, La, Gd, Lu) | | |
| MBO₃ | Pr, Ce, Bi | UV |
| (M = Y, La, Gd, Lu) | | |
| MB₃O₆ | Pr, Ce, Bi | UV |
| (M = Y, La, Gd, Lu) | | |
| MSO₄ | Nd, Pr, Ce, Pb | UV |
| (M = Sr, Ba) | | |
| LuPO₄ | Nd | 190 nm |
| YPO₄ | Nd | 190 nm |
| LaPO₄ | Nd | 185 nm |
| LaPO₄ | Pr | 225 nm |
| LuPO₄ | Pr | 233 nm |
| YPO₄ | Pr | 235 nm |
| YPO₄ | Bi | 240 nm |

The manufacturing method of the nanoparticles 22 is in principle not critical and thus can be any conventional production technique available. Several production techniques are known, whereby the selection of the most appropriate technique often depends on the specific components present in the nanoparticle 22, the size variance, purity, synthesis rate, etc. These techniques may be based on conventional techniques such as gas-phase synthesis, which may involve combustion flame, laser ablation, chemical vapour condensation, spray pyrolysis, electrospray and plasma spray, or sol-gel processing, which is a wet chemical synthesis approach based on gelation, precipitation and hydrothermal treatment. Other techniques such as sonochemical processing, micro-emulsion processing, high-energy ball milling, cavitation processing also may be used. It will be appreciated by a person skilled in the art that also other preparation techniques may be used. The preparation technique is only limited by the quality of the nanoparticles 22, i.e. the nanoparticles 22 obtained should preferably have sufficient homogeneity in emission characteristics. The emission spectrum is rather homogeneous, since it comprises a single emission band, which is rather narrow. The dispersion of the particle size distribution may preferably also be small, e.g. preferably the applied particles 22 only comprise particles between 10 and 20 nm in diameter. The homogeneity is specifically advantageous as usually one wants to know the dose delivered to the diseased tissue 20.

In FIG. 3 a scanning electron microscope picture of nanoparticles 22 is shown for the example of LaPO₄:Pr particles. From this picture it can be seen that the particles have a diameter of about 100 nm. The scale marker in the picture corresponds with a length of 1 μm.

In another embodiment, the nanoparticles 22 of the first embodiment can be brought immediately into the diseased tissue 20 and used for therapy instead of being injected into the blood. For example, a suspension of nanoparticles 22 can be injected into the tumour tissue 20 by a syringe. After e.g. 2 hours, the respective site is irradiated by a suitable source, e.g. x-rays with energy higher than 7 keV. The treatment can be repeated several times until the diseased tissue 20 is completely decomposed. The treatment can be the only treatment applied or it can be used in combination with other therapeutic techniques.

The solubility of a nanoparticle 22 increases typically with decreasing diameter. Therefore, the smaller the nanoparticles 22 are, the quicker they may be eliminated or cleared from the body. This size effect may be useful for adjustment of the clearance time.

The method of the present invention may also be applied is some specific cases where the diseased tissue or organ is taken out of the human body, treated with the method according to the present invention, and then put back into the body.

Furthermore, the method of the invention may be applied without the nanoparticles 22 being provided with specific binding sites. In this case, diffusion into healthy tissue and/or into other parts of the body might be inhibited by applying a coating or shell which limits the transport of the nanoparticle into the blood.

In a preferred embodiment, the host material preferentially is a trivalent phosphate. Trivalent cations have the advantage of having low solubility constants, e.g. pkₛₚ=22.4 for LaPO₄. Phosphate furthermore is hardly toxic as one of the blood buffers is the HPO₄²⁻/H₂PO₄⁻ ion couple. The toxicity of rare-earth phosphate compounds thus is low. These preferred nanoparticles 22 rely on an activator, e.g. Pr³⁺ and/or Nd³⁺ as activators, which have a very short radiative decay time, i.e. shorter than 100 ns. These short decay times restrict the energy migration to the nanoparticle 22 surface after the absorption process, which results in nanoparticle 22 phosphors having an energy efficiency close to that of micrometer particle phosphors. Energy migration is a process which occurs in any luminescent material after absorption of energy at an activator or sensitiser (dopant). The average distance of energy migration is dependent on the energy transfer efficiency from one ion to another one and on the decay constant of the excited state. The faster the decay of the excited ion is, the lower the probability is that energy transfer occurs. Thus, the average energy migration distance decreases with decreasing decay constant. Therefore, a short decay time of the activator (Pr³⁺, Nd³⁺, Ce³⁺, Bi³⁺) is required for small particles, since once the energy migrates to the surface, the excited state will be non-radiatively quenched. This is the reason why normal phosphor particles comprising slow activators, such as Eu³⁺ and Tb³⁺ must be in the micrometer range to prevent too much quenching and to achieve high quantum efficiencies. This means in turn that these slow activators yield nanomaterials with a low quantum efficiency.

The current embodiment also has the advantage of being of low cost, due to the application of cheap inorganic phosphates. Emission spectra of some exemplary phosphor materials are shown in FIG. 4 and FIG. 5. FIG. 4 shows the emission spectra of LaPO₄:Pr—indicated with the solid line—and YPO₄:Pr—indicated with the dashed line—nanoparticles 22 under high energy excitation. It can be seen that these phosphor materials emit in the region between 200 nm and 280 nm, LaPO₄:Pr having its highest emission peak position near 225 nm and YPO₄:Pr having its highest emission peak position near 233 μm. FIG. 5 shows the emission spectra for the same host materials having Nd as dopant. The emission for both phosphor materials ranges mainly between 200 nm and 175 nm.

Furthermore, small particles of phosphates are easily metabolised, i.e. dissolved within a couple of days and finally removed from the body.

Excitation of the luminescent nanoparticles 22 of the above embodiments is achieved by the application of x-ray radiation or high energy particles such as for example He-cores (α-radiation) or electrons (β-radiation). The x-ray cross section of the nanoparticles 22 is much higher than that of the surrounding tissue due to the high density of the nanoparticles 22. As an illustration, the density of some exemplary nanoparticles 22 is shown in Table 3. The nanoparticle 22 density is even much higher than that of standard radiosensitizers, such as halide substituted fluorescine or erythrosine. Typically, these organic radiosensitizers have a density between 1 and 2 g/cm³. The high x-ray cross-section has as a major advantage in that the applied x-ray dose can be significantly smaller than the dose required in standard radiation therapy. This leads to a decrease of damage to healthy tissue.

TABLE 3

| Phosphor material | Density [g/cm³] |
|---|---|
| LuPO₄: Nd | 6.5 |
| YPO₄: Nd | 3.7 |
| LaPO₄: Nd | 5.1 |
| LaPO₄: Pr | 5.1 |
| LuPO₄: Pr | 6.5 |
| YPO₄: Pr | 3.7 |
| YPO₄: Bi | 3.7 |

The absorption intensity as a function of the density of the tissue 20 is defined by formula (1)
Iₓ=I₀.e^{-(μ/ρ).ρ.x} (1)

wherein μ/ρ) is a constant, μ is the linear absorption coefficient, ρ is the material density and x is the penetration depth in the tissue 20. So, from this formula it can be seen that a high density leads to a large cross section for absorption of x-rays. As a result, the same therapeutic effect as that obtained by standard radiation therapy can be achieved with a much lower x-ray dose.

In a further embodiment, if the emitting material of the nanoparticle 22 is sensitive to hydrolysis or if there tends to be diffusion of components from the emitting material during transport, a coating 24 can be applied to the nanoparticles 22. This coating 24 completely encloses the emitting particle 22 and typically has a thickness of 1 to 200 nm, preferably between 5 to 20 nm. The coating 24 can consist of elementary Gold, SiO₂, a polyphosphate e.g. calcium polyphosphate, an amino acid e.g. aspartic acid, an organic polymer e.g. polyethylenglykol, polyvinylalcohol, polyamid, polyacrylate, polycarbamide, a Biopolymer e.g. a polysaccharide like Dextran, Xylan, Glykogen, Pectin, Cellulose or a Polypeptide like Collagene or Gluboline, Cystein e.g. Peptide with a large aspartic acid content or a Phospholipid. Besides avoiding hydrolysis and diffusion, depending on the type of coating 24 used, the coating 24 can improve the absorption of X-rays. This again can be advantageous for increasing the cross-section for absorption of the nanoparticles 22.

FIG. 6 shows an example of a schematic representation of an agent used according to the present invention, comprising a nanoparticle 22 which is a phosphor emitting in the VUV or UV-C region, a first coating 24 which is a coating 24 preventing hydrolysis and outdiffusion of components of the nanophosphor and a second coating of antibodies 26.

FIG. 7 shows a schematic diagram of the mechanism of the therapeutic treatment using VUV or UV-C emitting phosphate nanoparticles 22. The figure shows a nanoparticle phosphor 22 which is connected to an antibody 26 with a moiety 28. The moiety 28 can be e.g. an organic molecule comprising a carboxylic group. This may be an aromatic or aliphatic compound, e.g. olic acid or biotin. The latter is widely applied, since it binds strongly to avidin, which is recognised by certain types of antibodies. The antibody 26 can either bind to the surface of the cell and/or tissue 20 or to interior sites. The nanoparticle phosphor 22 is activated using x-ray radiation 30, which leads to VUV or UV-C emission 32 by the nanoparticle phosphor 22. The VUV or UV-C emission 32 destroys the cells, which are cells of diseased tissue 20 as the antibodies 26 preferentially bind to diseased tissue 20. The method can be applied solely or together with other therapeutic treatments.

In still another embodiment of the invention, the nanoparticles 22 may be preloaded with energy (activated) before implantation in the human body and energy may then be released in a later stadium. This phenomenon is called afterglow and is a known property of luminescent materials. Energy is stored in lattice defects at low temperature, for example at temperatures of below 250K, by X-ray irradiation. Initiation of emission may then occur at 37° C. in the human body, which results in the UV-C luminescence of the activator. An advantage of this embodiment is that activation, is separated from the medical treatment. Hence, in this embodiment, the human body does not have to be exposed to the X-ray irradiation.

Besides using the UV or VUV emission for destruction of cells, as described in the above embodiments, the emission can also be used for optical imaging. The UV-light can be detected endoscopically, i.e. using a long slender medical instrument for examining the interior of hollow organs including e.g. the lung, stomach, bladder and bowel. At locations where diseased tissue 20 is present, significantly higher emission intensity will be obtained because, due to the antibody-antigen reaction, the nanoparticles 22 will be mainly located at the diseased tissue 20. Due to the high sensitivity of the emitting nanoparticle 22 to the exciting X-ray radiation, medical imaging can be performed either to obtain the same sensitivity using a low X-ray dosage or to obtain an increased sensitivity using a high X-ray dosage. The possibility to obtain a higher detection sensitivity allows improved medical imaging. It is a specific advantage that a higher detection sensitivity can be obtained allowing a possible earlier detection of diseased tissue 20. This can be very important e.g. for early diagnosis of rapidly developing cancers. Medical imaging techniques can be used to study the extent of the damage caused by e.g. a cancer or for evaluating the effect of therapeutic treatments that already have been given.

In the following examples, two illustrations are given for the production of nanoparticles 22 for radiation therapy according to the present invention.

### EXAMPLE I

1.45 g Lu(CH₃COO)₃×H₂O, 1.64 g Si(OC₂H₅)₄ and 10 mg Pr(CH₃COO)₃×H₂O are suspended in 50 ml diethylene glycol. The suspension is stirred continuously and heated up to 140° C. Then, 0.5 ml of a 1M sodium hydroxide solution is added. Subsequently, the substance is heated for 8 hours at 190° C. After cooling down, a suspension remains comprising nanoscaled Lu₂SiO₅:Pr particles 22 (0.5 mol %) with a particle diameter of about 15 nm. The suspension is then centrifuged in order to separate the nanoscaled Lu₂SiO₅:Pr particles 22 from the solution. In a following step, the nanoscaled Lu₂SiO₅:Pr particles 22 are treated with a suitable washing process step, such as for example once again suspending the solid particles 22 in ethanol and/or acetone followed by again separating the particles 22 by centrifuging. In that way, the nanoparticles 22 formed can be separated from the first suspension and transferred into an aqueous solution (e.g. an isotonic solution respectively a phosphate buffer).

Starting from both the diethylene glycol based first suspension or from the second, aqueous suspension, nanoscaled Lu₂SiO₅:Pr particles 22 can further be modified. In that way, if to the resp. suspensions 10 ml of an aqueous solution, containing 100 mg Aspartic acid and 500 mg of Tetraethylorhtosilicate, is dripped during a period of 1 hour, a first cover 24 of SiO₂ containing Aspartic acid can be formed on the nanoparticle 22, the cover 24 having a thickness of about 15 nm. Finally, by adding 2 ml of an aqueous 10⁻⁴ solution of antibodies 26 such as for example Bevacizumab, or Histidin-modified antibodies such as for example Histidin-modified Bevacizumab, anti-bodies 26 can be attached to the Aspartic acid/SiO₂ layer by formation of amide bridges.

### EXAMPLE 2

6.97 g Lu(CH₃COO)₃×H₂O, 0.06 g Bi(CH₃COO)₃H₂O and 3.45 g NH₄H₂PO₄ are suspended in 500 ml diethylene glycol. The suspension is continuously stirred and heated up to 140° C. Then, 2.0 ml of a 2 M sodium hydroxide solution is added. Subsequently, the suspension is heated for 4 hours at 180° C. The remaining suspension comprises nanoscaled LuPO₄:Bi (1 mol %) particles 22 with a particle diameter of 30 nm. The nanoscaled particles 22 can be transferred to an aqueous solution by separating them from this first suspension by centrifuging the suspension followed by a suitable washing process, such as for example once again suspending the solid solution in ethanol and/or acetone and again centrifuging.

Starting from either the diethylene glycol based first suspension or from the second aqueous suspension, nanoscaled LuPO₄:Bi particles 22 can further be modified. To the first or second suspension 20 ml of an aqueous 10⁻³ M solution of Aspartic acid modified Dextran is dripped. In that way, a first cover 24 of Dextran can be formed on the nanoparticle 22, the cover 24 of Dextran having a thickness of about 20 nm. Finally, by adding 3 ml of an aqueous 10⁻⁴ solution of antibodies 26 such as for example anti-CEA or of Histidin-modified antibodies such as for example Histidin-modified anti-CEA, antibodies 26 can be attached to the Aspartic acid/Dextran layer by formation of amide bridges.

It is to be understood that although preferred embodiments, specific constructions and configurations, as well as materials, have been discussed herein for devices according to the present invention, various changes or modifications in form and detail may be made without departing from the scope and spirit of this invention.

1. Nanoparticles for use in imaging or in radiation treatment of biological material, the nanoparticles comprising a VUV or UV-C emitting material which absorbs high energy radiation and emits VUV or UV-C radiation, said nanoparticles being conjugated to a bio-target specific agent.

2. Nanoparticles as claimed in claim 1, for use in radiation therapy.

3. Nanoparticles as claimed in claim 1, wherein the high energy radiation is X-rays.

4. Nanoparticles as claimed in claim 1, wherein said bio-target specific agents are antibodies or antibody fragments.

5. Nanoparticles as claimed in claim 4, wherein the antibodies or antibody fragments have a specificity for a diseased tissue.

6. Nanoparticles as claimed in claim 1, wherein the UV emitting material of the nanoparticles is provided with a covering layer.

7. Nanoparticles as claimed in claim 6, wherein the covering layer prevents hydrolysis of the UV emitting material.

8. Nanoparticles as claimed in claim 1, wherein the VUV or UV-C emitting material is one or more substances selected from the group: -, M₂SiO₅:X, MAlO₃:X, M₃Al₅O₁₂:X, MPO₄:X, MBO₃:X, MB₃O₆:X with M=Y, La, Gd, Lu, and X=Pr, Ce, Bi, Nd or any of MM′O₃:X with M=Y, La, Gd, Lu, M′=Y, La, Gd, Lu, Bi and X=Pr, Ce, Bi or any of MSO₄:Z with M=Sr, Ca and Z=Nd, Pr, Ce, Pb or any of LuPO₄:Nd, YPO₄:Nd, LaPO₄:Nd, LaPO₄:Pr, LuPO₄:Pr, YPO₄:Pr, YPO₄:Bi.

9. Nanoparticles as claimed in claim 1, wherein the VUV or UV-C emitting material is a trivalent phosphate.

10. Nanoparticles as claimed in claim 1, wherein the nanoparticles are doped with an activator.

11. Nanoparticles according to claim 10, wherein the activator has a decay time shorter than 100 ns.

12. Nanoparticles as claimed in claim 10, wherein said activator is Pr³⁺ or Nd³⁺.

13. The use of nanoparticles as an imaging agent or a radiation treatment agent, the nanoparticles comprising a VUV or UV-C emitting material which absorbs high energy radiation and emits VUV or UV-C radiation.

14. The use of claim 13, in the manufacture of an imaging agent or a radiation therapy agent.

15. The use as claimed in claim 13, wherein the high energy radiation is X-rays.

16. The use as claimed in claim 13, said nanoparticles being conjugated to a bio-target specific agent.

17. The use as claimed in claim 16, wherein said bio-target specific agents are antibodies or antibody fragments.

18. The use as claimed in claim 17, wherein the antibodies or antibody fragments have a specificity for the bio-target.

19. The use as claimed in claim 13, wherein the UV emitting material of the nanoparticles is provided with a covering layer.

20. The use as claimed in claim 19, wherein the covering layer prevents hydrolysis of said UV emitting material.

21. The use as claimed in claim 13, wherein the VUV or UV-C emitting material is one or more substances selected from the group: M₂SiO₅:X, MAlO₃:X, M₃Al₅O₁₂:X, MPO₄:X, MBO₃:X, MB₃O₆:X with M=Y, La, Gd, Lu, and X=Pr, Ce, Bi, Nd or any of MM′O₃:X with M=Y, La, Gd, Lu, Bi, M′=Y, La, Gd, Lu, and X=Pr, Ce, Bi or any of MSO₄:Z with M=Sr, Ca and Z=Nd, Pr, Ce, Pb or any of LuPO₄:Nd, YPO₄:Nd, LaPO₄:Nd, LaPO₄:Pr, LuPO₄:Pr, YPO₄:Pr, YPO₄:Bi.

22. The use as claimed in claim 13, wherein the UV emitting material is a trivalent phosphate.

23. The use as claimed in claim 13, wherein the nanoparticles are doped with an activator.

24. The use as claimed in claim 23, wherein said activator is Pr³⁺ or Nd³⁺.

25. A method of treatment of a human or an animal patient by: providing nanoparticles according to claim 1, administering the nanoparticles to the patient, and irradiating the patient with high energy radiation.
